(19) United States
(12) Patent Application Publication
(10) Pub. No.: US 2024/0336483 A1
(43) Pub. Date: Oct. 10, 2024

(54) APPARATUS AND METHOD FOR PREPARING HYDROXYAPATITE SUSPENSION

(71) Applicant: Brainy Inc., Chiba (JP)

(72) Inventors: Nobukiyo Tanaka, Okinawa (JP); Masashi Tanaka, Chiba (JP)

(73) Assignee: Brainy Inc., Chiba (JP)

(21) Appl. No.: 18/744,899

(22) Filed: Jun. 17, 2024

Related U.S. Application Data
(63) Continuation of application No. PCT/JP2022/017239, filed on Apr. 7, 2022.

(51) Int. Cl. C01B 25/32

(52) U.S. Cl. CPC C01B 25/322

(57) ABSTRACT

A hydroxyapatite suspension preparation apparatus includes a container, first and second injection devices, a pH meter, a controller, and a pouring device. The injection device injects calcium hydroxide into the container. The second injection device injects phosphoric acid into a solution contained in the container. The pH meter measures a pH of the solution in the container. The controller controls concentration of the calcium hydroxide injected by the first injection device and at least one of concentration and an injection rate of the phosphoric acid injected by the second injection device so that the pH is within a predetermined range. The pouring device pours hydroxyapatite suspension concentrated by spontaneous sedimentation.

### CROSS-REFERENCE TO RELATED APPLICATIONS

This application is a Continuation Application of PCT Application No. PCT/JP2022/017239, filed Apr. 7, 2022, the entire contents of which are incorporated herein by reference.

### BACKGROUND OF THE INVENTION

### 1. Field of the Invention

The present invention relates generally to an apparatus and method for preparing hydroxyapatite suspension.

### 2. Description of the Related Art

Hydroxyapatite (hereinafter referred to as HA) is represented, for example, by Ca₁₀(PO₄)₆(OH)₂. An example of a method for preparing HA is a solution method. Generally, HA is transported, sold, and utilized in dry powder form. HA aggregates when dried. The particle size of the aggregated HA becomes micro-sized.

### BRIEF SUMMARY OF THE INVENTION

An embodiment of the invention provides an apparatus and method for preparing suspension containing HA with a small particle size.

A HA suspension preparation apparatus according to an embodiment of the invention includes a container, a first injection device, a second injection device, a pH meter, a controller, and a pouring device. The first injection device injects calcium hydroxide into the container. The second injection device injects phosphoric acid into a solution contained in the container. The pH meter measures a pH of the solution in the container. The controller that controls concentration of the calcium hydroxide injected by the first injection device and at least one of concentration and an injection rate of the phosphoric acid injected by the second injection device so that the pH measured by the pH meter is within a predetermined range. The pouring device pours hydroxyapatite suspension concentrated by spontaneous sedimentation of the solution in the container out of the container.

According to the embodiment of the present invention, suspension containing HA with a small particle size can be prepared.

Additional objects and advantages of the invention will be set forth in the description which follows, and in part will be obvious from the description, or may be learned by practice of the invention. The objects and advantages of the invention may be realized and obtained by means of the instrumentalities and combinations particularly pointed out hereinafter.

### BRIEF DESCRIPTION OF THE SEVERAL VIEWS OF THE DRAWING

The accompanying drawings, which are incorporated in and constitute a part of the specification, illustrate embodiments of the invention, and together with the general description given above and the detailed description of the embodiments given below, serve to explain the principles of the invention.

FIG. 1 is a block diagram showing an example of a configuration of a preparation apparatus according to a first embodiment.

FIG. 2 is a conceptual diagram showing an example of a mixed state of calcium hydroxide and phosphoric acid by the preparation apparatus according to the first embodiment.

FIG. 3 is a flowchart showing an example of a suspension preparing method executed by the preparation device according to the first embodiment.

FIG. 4 is a conceptual diagram showing an example of spontaneous sedimentation in the suspension preparing method according to the first embodiment.

FIG. 5 is a view showing an example of experimental results of a general viable bacteria test.

FIG. 6 is a view showing an example of experimental results of a fungal test.

FIG. 7 is a view showing an example of experimental results of autoclave sterilization.

FIG. 8 is a view showing an example of experimental results of a general viable bacteria test.

FIG. 9 is a graph showing an example of a relationship between pH and the growth rate of viable bacteria obtained based on the results of a general viable bacteria test.

FIG. 10 is a conceptual diagram showing an example of a configuration of an application apparatus for applying silver-containing HA to a nonwoven fabric according to a second embodiment.

FIG. 11 is a side view showing an example of a shower head and an ultrasonic transducer.

FIG. 12 is a perspective view showing an example of an upper surface of a support stand.

FIG. 13 is a flowchart showing an example of a method of applying silver-containing HA to the nonwoven fabric performed by the application apparatus according to the second embodiment.

### DETAILED DESCRIPTION OF THE INVENTION

Embodiments of the present invention will be described below with reference to the accompanying drawings. Note that, in the following description, functions and components that are almost or substantially the same are given the same symbols, and explanations thereof are omitted or given only when necessary.

### First Embodiment

In a first embodiment, an apparatus and method for preparing HA suspension that contains HA with a small particle size, has high transport efficiency, and is highly effective in controlling the number of bacteria will be described using a solution method.

The HA contained in the HA suspension prepared by the first embodiment can maintain its nanosize.

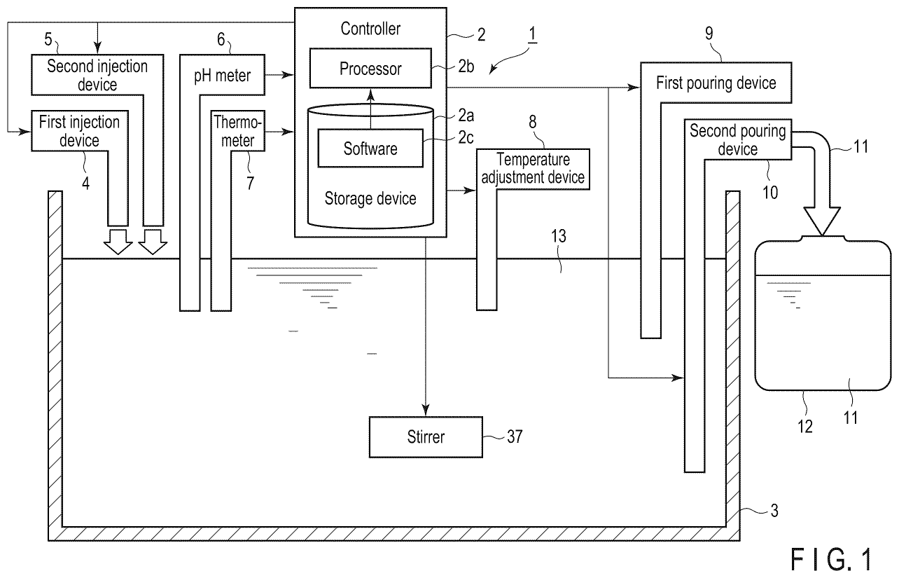

FIG. 1 is a block diagram showing an example of a configuration of a preparation apparatus 1 according to the first embodiment.

The preparation apparatus 1 includes a controller 2, a first container 3, a first injection device 4, a second injection device 5, a pH (hydrogen ion concentration index) meter 6, a thermometer 7, a temperature adjustment device 8, a stirrer 37, a first pouring device 9, and a second pouring device 10. HA suspension 11 prepared and concentrated by the preparation apparatus 1 is contained in a second container 12.

Note that the various devices configuring the preparation apparatus 1 may be combined as appropriate. For example, the pH meter 6 and the thermometer 7 may be one device. For example, the first pouring device 9 and the second pouring device 10 may be one pump.

The various devices configuring the preparation apparatus 1 may also be separated as appropriate. For example, the first container 3 may be divided into a container used for dropping phosphoric acid into calcium hydroxide and a container for spontaneous sedimentation of solution 13 which is a mixture of calcium hydroxide and phosphoric acid.

The controller 2 receives a signal indicating a pH measured by the pH meter 6 and a signal indicating a temperature measured by the thermometer 7.

Based on the pH indicated by the signal received from the pH meter 6 and the temperature indicated by the signal received from the thermometer 7, the controller 2 controls the concentration of calcium hydroxide to be injected into the first container 3 by the first injection device 4, at least one of the concentration and the injection rate (e.g., dropping rate) of phosphoric acid to be injected (e.g., dropped) into the first container 3 by the second injection device 5, the pH of the solution 13 (synthesis pH), and the temperature management of the solution 13 during HA synthesis and spontaneous sedimentation by the temperature adjustment device 8.

The controller 2 may perform control to intentionally prepare calcium-deficient HA or calcium-deficient HA containing antimicrobial heavy metals by additionally dropping phosphoric acid after dropping phosphoric acid and lowering the pH of the solution 13.

In the first embodiment, the controller 2 controls the concentration of calcium hydroxide so that the concentration of calcium hydroxide is within a first range. The first range may be, for example, 0.01% or more and 50% or less.

The controller 2 controls the concentration of phosphoric acid so that the concentration of phosphoric acid is within a second range. The second range may be, for example, 0.01% or more and 50% or less.

The controller 2 controls the injection rate of phosphoric acid so that the injection rate of phosphoric acid with respect to HA production amount is within a third range. The third range may be, for example, 0.01 ml/min/g or more and 100 ml/min/g or less. The third range may preferably be, such as, 0.1 ml/min/g or more and 10 ml/min/g or less.

The controller 2 controls the injection amount of at least one of calcium hydroxide and phosphoric acid so that the pH of the solution 13 in the first container 3 is within a fourth range. The fourth range may be, for example, 5 or more and 13 or less.

The controller 2 controls the temperature of the solution 13 so that the synthesis temperature at which the calcium hydroxide and phosphoric acid are mixed to synthesize the HA suspension and the temperature at which the calcium hydroxide and phosphoric acid are reacting are within a fifth range. The fifth range may be, for example, 5° C. or higher and 50° C. or lower. The fifth range may preferably be, such as, 5° C. or higher and 30° C. or lower. Although the temperature of the solution 13 tends to increase due to reaction heat during the HA preparation reaction, the operation of the controller 2 and the temperature adjustment device 8 can achieve conditions suitable for HA preparation.

The controller 2 may, for example, include a storage device 2a and a processor 2b. In this case, the storage device 2a stores software 2c. The storage device 2a may include a non-temporary storage device and a temporary storage device such as a cache memory. The software 2c may include a program and data. The processor 2b executes the software 2c stored in the storage device 2a and performs various controls for preparing the HA suspension 11.

When mixing calcium hydroxide and phosphoric acid (HA synthesis), the controller 2 executes control to cause the stirrer 37 to stir the solution 13 in the container 3. More specifically, the controller 2 transmits a signal indicating a rotation speed to the stirrer 37 when the second injection device 5 performs the dropping process of phosphoric acid. For example, the controller 2 transmits a signal to the stirrer 37 to rotate the stirrer 37 at, for example, 50 rpm or higher and 1000 rpm or lower. More preferably, the controller 2 transmits a signal to the stirrer 37 to rotate the stirrer 37 at, for example, 100 rpm or higher and 500 rpm or lower.

After pouring out the supernatant portion of the solution 13 from the first container 3 and before pouring out the concentrated HA suspension 11 from the first container 3, the controller 2 executes control to cause the stirrer 37 to stir the concentrated HA suspension 11 in the first container 3. More specifically, the controller 2 transmits instructions indicating the rotation speed to the stirrer 37 prior to pouring out the concentrated HA suspension 11. For example, the controller 2 transmits a signal to the stirrer 37 to rotate the stirrer 37 at, for example, 50 rpm or higher and 1000 rpm or lower. More preferably, the controller 2 transmits a signal to the stirrer 37 to rotate the stirrer 37 at, for example, 100 rpm or higher and 500 rpm or lower. This stirring by the stirrer 37 allows the concentration of the concentrated HA suspension 11 to be uniform.

The controller 2 instructs the first pouring device 9 to pour out the supernatant portion of the solution 13 contained in the first container 3.

The controller 2 instructs the second pouring device 10 to pour out the settled portion (i.e., the concentrated HA suspension 11) of the solution 13 contained in the first container 3.

The first container 3 contains calcium hydroxide (solution) injected from the first injection device 4.

The first container 3 receives phosphoric acid (liquid) injected (e.g., dropped) by the second injection device 5.

In the first container 3, calcium hydroxide and phosphoric acid react to prepare (synthesize) the solution 13 including HA.

The first injection device 4 receives a signal indicating the concentration of calcium hydroxide from the controller 2. Based on the received signal, the first injection device 4 adjusts the concentration of calcium hydroxide and injects the calcium hydroxide with the adjusted concentration into the first container 3. In the first embodiment, the purity of the calcium hydroxide may be 90% or higher and 100% or lower.

The first injection device 4 may inject a liquid mixture of calcium hydroxide and a compound containing an antimicrobial heavy metal into the first container 3. In the first embodiment, the antimicrobial heavy metal may be, for example, silver, copper, palladium, platinum, cadmium, nickel, cobalt, zinc, manganese, thallium, lead, or mercury.

The second injection device 5 receives a signal indicating the concentration of phosphoric acid and a signal indicating the injection rate of phosphoric acid from the controller 2. The second injection device 5 drops phosphoric acid at the concentration indicated by the received signal into the solution 13 containing calcium hydroxide stored in the first container 3 at the injection rate indicated by the received signal.

The second injection device 5 may drop a liquid mixture of phosphoric acid and a compound containing an antimicrobial heavy metal into the solution 13 in the first container 3.

The pH meter 6 measures the pH of the solution 13 stored in the first container 3 and transmits a signal indicating the pH to the controller 2.

The thermometer 7 measures the temperature of the solution 13 stored in the first container 3 and transmits a signal indicating the temperature to the controller 2.

The temperature adjustment device 8 receives the signal indicating the temperature from the controller 2. The temperature adjustment device 8 executes temperature management (e.g., heating or cooling) of the solution 13 in the first container 3 so that the solution 13 in the first container 3 becomes the temperature indicated by the received signal during mixing of calcium hydroxide and phosphoric acid (during HA synthesis) and during execution of spontaneous sedimentation on the solution 13.

The stirrer 37 receives a signal indicating the rotation speed from the controller 2. The stirrer 37 stirs the solution 13 in the container 3 according to the rotation speed indicated by the received signal. The stirrer 37 stirs the solution 13 during the dropping of phosphoric acid (during HA synthesis). The stirrer 37 also stirs the concentrated HA suspension 11 before pouring out the concentrated HA suspension 11. There is a concentration gradient in the settled portion after the supernatant portion is poured out. In the first embodiment, the concentration of the settled portion can be made uniform by stirring the settled portion.

The first pouring device 9 receives a signal indicating a pouring instruction from the controller 2. Based on the received signal, the first pouring device 9 pours out the supernatant portion of the solution 13 from the first container 3, leaving the settled portion of the solution 13 (i.e., the concentrated HA suspension 11) in the first container 3.

An inlet of the first pouring device 9 can be moved up and down according to a boundary position between the supernatant portion and the settled portion. More specifically, the height of the inlet of the first pouring device 9 is adjusted to be above the boundary position between the supernatant portion and the settled portion. This allows the supernatant portion to be efficiently poured out and the settled portion to remain in the first container 3.

The second pouring device 10 receives a signal indicating a pouring instruction from the controller 2. Based on the received signal, the second pouring device 10 injects the settled portion of the solution 13 in the first container 3 into the second container 12 for transport as a concentrated HA suspension.

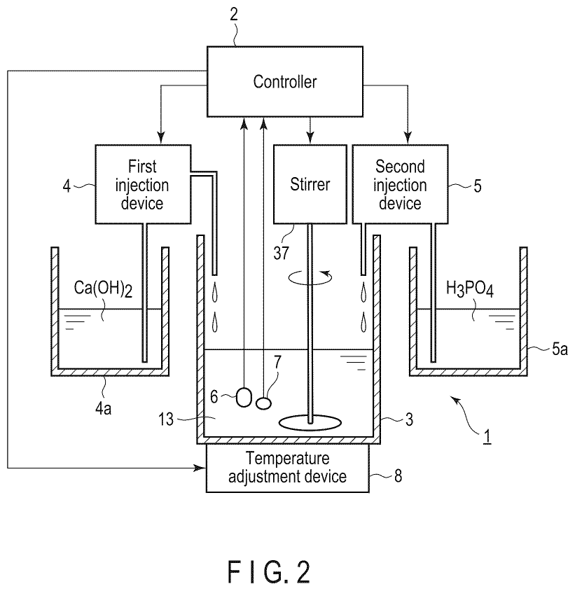

FIG. 2 is a conceptual diagram showing an example of a mixed state of calcium hydroxide and phosphoric acid (HA synthesis state) by the preparation apparatus 1 according to the first embodiment. In this FIG. 2, the first pouring device 9 and the second pouring device 10 of the preparation apparatus 1 are omitted.

A third container 4a contains a solution of calcium hydroxide. The first injection device 4 adjusts the calcium hydroxide to the concentration indicated by the signal received from the controller 2, and injects the calcium hydroxide with the adjusted concentration into the first container 3.

A fourth container 5a contains a solution of phosphoric acid. The second injection device 5 adjusts the phosphoric acid to the concentration indicated by the signal received from the controller 2, and drops the phosphoric acid with the adjusted concentration into the first container 3 at the dropping rate indicated by the signal received from the controller 2.

The controller 2 receives a signal indicating the pH of the solution 13 from the pH meter 6, and based on the pH of the solution 13, controls the operation of the first injection device 4 and the second injection device 5, and the stirring process of the stirrer 37.

The controller 2 receives a signal indicating the temperature of the solution 13 from the thermometer 7, and controls the operation of the temperature adjustment device 8 based on the temperature of the solution 13.

In the first embodiment, the first injection device 4 and the second injection device 5 may be, for example, tube pumps.

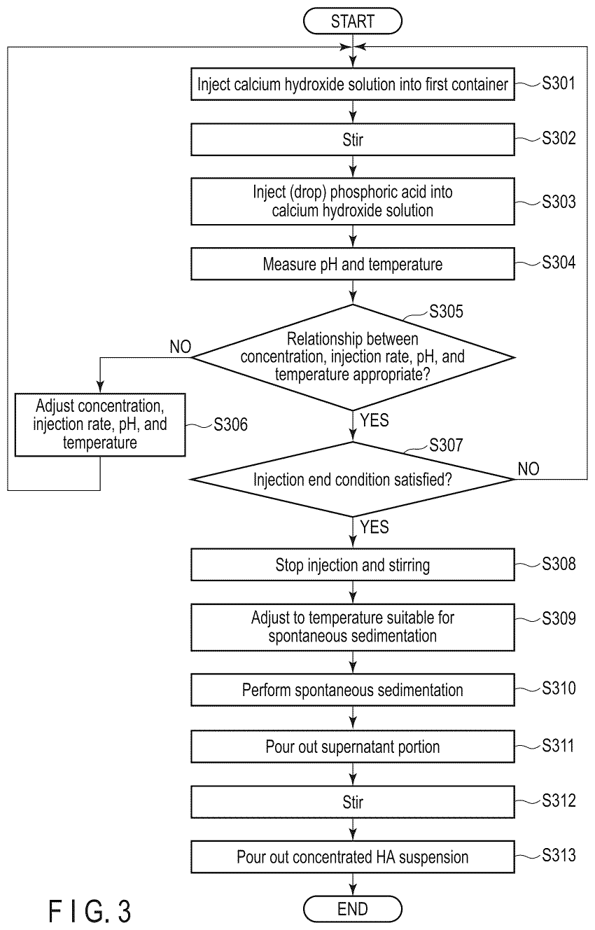

FIG. 3 is a flowchart showing an example of a method of preparing the HA suspension 11 executed by the preparation device 1 according to the first embodiment. The preparation method shown in FIG. 3 is executed in accordance with the control by the controller 2.

In S301, the first injection device 4 injects calcium hydroxide into the first container 3 at the concentration specified by the controller 2.

In S302, the stirrer 37 operates at the rotation speed specified by the controller 2 to stir the solution 13 in the first container 3.

In S303, the second injection device 5 drops phosphoric acid into the solution 13 (calcium hydroxide) in the first container 3 at the concentration and injection rate specified by the controller 2.

In S304, the pH meter 6 measures the pH of the solution 13 in the first container 3 and transmits a signal indicating the pH to the controller 2, and the thermometer 7 measures the temperature of the solution 13 in the first container 3 and transmits a signal indicating the temperature to the controller 2.

In S305, the controller 2 determines whether or not the relationship between the concentration of calcium hydroxide, the concentration and injection rate of phosphoric acid, the pH of the solution 13 in the first container 3, and the temperature of the solution 13 is appropriate. This appropriate relationship between the concentration of the various materials, injection rate, pH, and temperature will be explained later.

In a case where the controller 2 determines that the relationship between the concentration, injection rate, pH, and temperature is inappropriate, in S306, the controller 2 determines a new calcium hydroxide concentration, a new phosphoric acid concentration and a new phosphoric acid injection rate, a new pH, and a new temperature of the solution 13 in the first container 3. The controller 2 then transmits a signal indicating the new calcium hydroxide concentration to the first injection device 4. The first injection device 4 adjusts the concentration of calcium hydroxide based on the signal received from the controller 2. The controller 2 transmits a signal indicating the new phosphoric acid concentration and a signal indicating the new phosphoric acid injection rate of to the second injection device 5. The second injection device 5 adjusts the concentration and injection rate of the phosphoric acid based on the signal received from the controller 2. The controller 2 transmits a signal indicating the new temperature to the temperature adjustment device 8. The temperature adjustment device 8 adjusts the temperature of the solution 13 based on the signal received from the controller 2. The process then returns to S301.

Note that, in S306 above of the first embodiment, the controller 2 does not have to determine the new calcium hydroxide concentration and does not have to transmit a signal indicating the new calcium hydroxide concentration to the first injection device 4. In this case, the process returns to S302.

In a case where the controller 2 determines that the relationship between the concentration, injection rate, pH, and temperature is appropriate in S305 above, in S307, the controller 2 determines whether or not the end condition of the operation to drop phosphoric acid on calcium hydroxide (condition for ending dropping) is satisfied.

The condition for ending dropping may be, for example, that the pH of the solution 13 in the first container 3 has reached a target range (target pH).

After the pH of the solution 13 reaches the target pH, the pH of the solution 13 may increase after a while. Therefore, even after the pH of the solution 13 reaches the target pH, the controller 2 causes the second injection device 5 to drop phosphoric acid again as appropriate thereafter, and, in the case where the pH of the solution 13 stabilizes, the controller 2 may determine that the condition for ending dropping is satisfied.

The condition for ending dropping may be, for example, that the amount of solution 13 in the first container 3 exceeds a threshold value.

The controller 2 may intentionally prepare calcium-deficient HA or calcium-deficient HA containing antimicrobial heavy metals by lowering the pH of the solution 13 by additional drops of phosphoric acid after dropping the phosphoric acid.

In the case where the controller 2 determines that the condition for ending is not satisfied, the process returns to S301. Note that, in the case where the controller 2 does not determine the new calcium hydroxide concentration in S306 of the first embodiment, the process may return to S302.

In the case where the controller 2 determines that the ending condition is satisfied, in S308, the controller 2 transmits a stop signal to the first injection device 4, the second injection device 5, and the stirrer 37. The first injection device 4 and the second injection device 5 stop the injection operation according to the signal received from the controller 2. The stirrer 37 stops the stirring operation according to the signal received from the controller 2.

In S309, the controller 2 transmits a signal indicating a temperature suitable for spontaneous sedimentation to the temperature adjustment device 8. The temperature adjustment device 8 adjusts the temperature of the solution 13 based on the signal received from the controller 2.

In step S310, the spontaneous sedimentation of the solution 13 in the first container 3 is carried out for a predetermined period of time or longer.

In S311, the first pouring device 9 pours out the supernatant portion of the solution 13 in the first container 3 according to the control by the controller 2, leaving the settled portion in the first container 3.

In S312, the stirrer 37 stirs the concentrated HA suspension 11, which is the settled portion, in the first container 3 according to the control by the controller 2.

In S313, the second pouring device 10 pours out the concentrated HA suspension 11 in the first container 3 according to the control by the controller 2, and stores it in the second container 12.

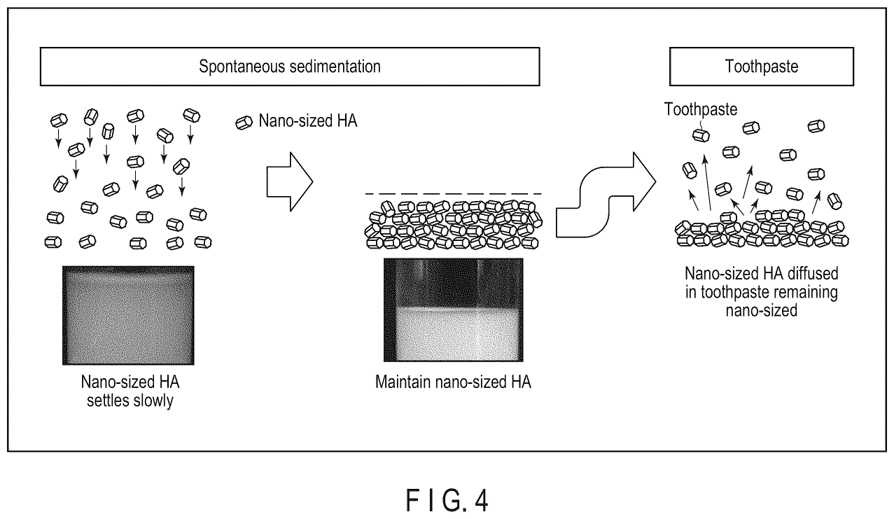

FIG. 4 is a conceptual diagram showing an example of spontaneous sedimentation in the method of preparing the HA suspension 11 according to the first embodiment.

The solution 13 containing HA prepared in the first container 3 is subjected to spontaneous sedimentation for a predetermined period of time. The predetermined period of time for spontaneous sedimentation may be, as a result of experiments, for example, from 1 to 60 days, or more preferably from 9 to 28 days.

The nano-sized HA in the solution 13 settles slowly. When the supernatant portion of the solution 13 is removed, a concentrated HA suspension 11, which is the settled portion of the solution 13, remains. The HA contained in this HA suspension 11 maintains its nanosize.

By mixing the concentrated HA suspension 11 with toothpaste, a toothpaste with diffused nano-sized HA can be prepared.

In the following, the relationship between the concentration of calcium hydroxide, the injection rate of phosphoric acid, and the temperature of the solution 13 in the first embodiment is explained.

The solution method, which is one of the methods of HA preparation, can reduce the particle size of HA compared to hydrothermal synthesis and dry synthesis, and is suitable for mass production.

The solution method includes the ammonia-based solution method, in which ammonium phosphate is added to calcium nitrate for synthesis, and the calcium hydroxide-based solution method, in which phosphoric acid is added to calcium hydroxide. The ammonia-based solution method uses ammonia, therefore, requires washing. This washing causes a loss of HA yield in the ammonia-based solution method. The calcium hydroxide solution method does not require washing because there are no byproducts. Therefore, the calcium hydroxide solution method can achieve a higher HA yield rate than the ammonia-based solution method.

Here, the HA yield rate is the ratio of the actual HA production amount to the calculated HA synthesis amount calculated from the raw materials.

In the case of selling and using HA in dry powder form, even if nano-sized HA is prepared, the HA may aggregate due to drying, resulting in micro-sized HA.

Therefore, in the first embodiment, the calcium hydroxide-based solution method is used to prepare a concentrated HA suspension 11 with a small particle size and excellent transport efficiency and bacterial count control. In the following, various conditions (effects of raw material concentration, injection rate, temperature, pH, etc.) applied in the apparatus 1 and method for preparing the HA suspension 11 according to the first embodiment will be described.

In the first embodiment, bacterial count control is achieved by adjusting the pH of the solution 13 in the first container 3.

In the first embodiment, the solution 13 is concentrated using spontaneous sedimentation, instead of a centrifuge and drying, to maintain the HA particle size small and to increase transport efficiency.

Furthermore, the first embodiment describes a preferred spontaneous sedimentation temperature and period for concentration.

In the first embodiment, experiments were conducted to dry or heat the suspension and analyze and identify the products. Tricalcium phosphate (TCP) or calcium oxide (CaO) was detected in the product obtained from this experiment. The reason why the product contains tricalcium phosphate is considered to be due to the presence of Ca-deficient HA in the suspension. It is believed that the product contains calcium oxide due to the presence of calcium hydroxide in the suspension. In order to maximize the amount of HA prepared, it is important to control the suspension to an appropriate pH.

Additionally, the pH of the product varies with temperature. As the temperature decreases, the pH in an optimum condition also decreases.

Table 1 shows examples of experimental results of the relationship between temperature, pH, and HA yield rate.

TABLE 1

| | |
|---|---|
| 25.5° C. | 19.1° C. |

| pH | HA | TCP | CaO | HA | TCP | CaO |
|---|---|---|---|---|---|---|
| 7 | 73 | 27 | 0 | 55 | 45 | 0 |
| 7.5 | 77 | 23 | 0 | 70 | 30 | 0 |
| 8 | 88 | 12 | 0 | 74 | 26 | 0 |
| 8.5 | 99.9 | 0.1 | 0 | 80 | 20 | 0 |
| 9 | 100 | 0 | 0 | 93 | 7 | 0 |
| 9.5 | 99.5 | 0 | 0.5 | 99.9 | 0.1 | 0 |

From Table 1, it can be seen that the pH at which the HA yield rate increases varies with temperature. In Table 1, at a temperature of 25.5° C., the appropriate pH at which the HA yield rate increases is 8.5 to 9.0. In contrast, at 19.1° C., the pH at which the HA yield rate increases rises to 9.5.

Table 2 shows examples of experimental results of the relationship between pH and calcium hydroxide prity.

TABLE 2

| | |
|---|---|
| High prity | |
| Ca(OH)₂ 99.9% | HA Ca(OH)₂ 96-97% |

| pH | HA | TCP | CaO | HA | TCP | CaO |
|---|---|---|---|---|---|---|
| 10 | — | — | — | 99.9 | 0 | 0.1 |
| 9.5 | 99 | 0 | 1 | 99.9 | 0.1 | 0 |
| 9 | 99.2 | 0 | 0.8 | 99.9 | 0.1 | 0 |
| 8.5 | 99.5 | 0 | 0.5 | 93 | 7 | 0 |
| 8 | 100 | 0 | 0 | 80 | 20 | 0 |
| 7.5 | 100 | 0 | 0 | 74 | 26 | 0 |
| 7 | 99.9 | 0.1 | 0 | 70 | 30 | 0 |
| 6.5 | 87 | 13 | 0 | 55 | 45 | 0 |
| 6 | 75 | 25 | 0 | — | — | — |

In the case where the calcium hydroxide prity is increased from 96% to 97% to a very high prity of 99.9%, the appropriate pH at which the HA yield rate becomes large decreases by about 1.5.

Table 3 shows examples of experimental results of the relationship between the injection rate of phosphoric acid during synthesis and HA particle size. This Table 3 shows the relationship between the injection rate of phosphoric acid and the HA particle size when the HA yield is 50 g.

TABLE 3

| Injection rate | HA particle |
|---|---|
| (ml/min) | diameter (μm) |
| 4 | 10-30 |
| 8 | 10-50 |
| 16 | 10-50 |
| 32 | 60-100 |

The particle size of HA increases with an increase in the injection rate of phosphoric acid during synthesis.

Table 4 shows examples of experimental results of the relationship between the HA yield, the injection rate of phosphoric acid, and the HA particle size.

TABLE 4

| HA yield | Injection rate | HA particle |
|---|---|---|
| (g) | (ml/min) | diameter (μm) |
| 25 | 4 | 10-40 |
| 50 | 8 | 10-50 |
| 100 | 16 | 10-50 |

From this Table 4, it can be seen that for the same particle diameter, the injection rate of phosphoric acid can be increased with an increase in the HA yield. For example, if the HA yield is doubled, the injection rate of phosphoric acid can also be doubled.

Table 5 shows a first example of experimental results of the relationship between calcium hydroxide, phosphoric acid, and particle diameter of HA. In this Table 5, the dilution ratio of phosphoric acid is held constant.

TABLE 5

| Ca(OH)₂ | H₃PO₄ | |
|---|---|---|
| Dilution ratio | Dilution ratio | HA particle |
| (Magnification) | (Magnification) | diameter (μm) |
| 20 | 11 | 50-200 |
| 40 | 11 | 30-70 |

From Table 5, the higher the dilution ratio of calcium hydroxide, the smaller the particle diameter of HA can be made.

Table 6 shows a second example of experimental results of the relationship between calcium hydroxide, phosphoric acid, and particle diameter of HA. In this Table 6, the dilution ratio of calcium hydroxide is held constant.

TABLE 6

| Ca(OH)₂ | H₃PO₄ | |
|---|---|---|
| Dilution ratio | Dilution ratio | HA particle |
| (Magnification) | (Magnification) | diameter (μm) |
| 10 | 11 | 40-100 |
| 10 | 23 | 10-50 |

From Table 6, it can be seen that the higher the dilution ratio of phosphoric acid, the smaller the particle diameter of HA can be made.

Table 7 shows a third example of experimental results of the relationship between the dilution ratio of calcium hydroxide, the dilution ratio of phosphoric acid, and the particle diameter of HA.

TABLE 7

| Ca(OH)₂ | H₃PO₄ | |
|---|---|---|
| Dilution ratio | Dilution ratio | HA particle |
| (Magnification) | (Magnification) | diameter (μm) |
| 100 | 11 | 1-10 |
| 100 | 23 | 1-10 |
| 100 | 47 | 1-5 |
| 200 | 47 | <1 |

Fromm Table 7, it can be seen that the higher the dilution ratio of calcium hydroxide and the dilution ratio of phosphoric acid, the smaller the particle diameter of HA can be made.

From Tables 3 to 7 above, the controller 2, for example, executes control so that the dilution ratio of calcium hydroxide is higher than a first threshold value, the dilution ratio of phosphoric acid is higher than a second threshold value, and the injection rate of phosphoric acid is slower than a third threshold value. The first threshold value may be, for example, any value within the range of 0.01% or higher and 50% or lower. The second threshold value may be, for example, any value within the range of 0.01% or higher and 50% or lower. The third threshold value is a value related to the injection rate with respect to the amount of HA prepared, and may be, for example, any value within the range of 0.01 ml/min/g or higher and 10 ml/min/g or lower.

This allows the particle size of the HA contained in the solution 13 and the HA suspension 11 to be as small as, for example, nano-sized.

In the first embodiment, the solution 13 prepared in the first container 3 is concentrated using spontaneous sedimentation without drying, and the concentrated HA suspension 11 is contained in the second container 12 and then transported and utilized.

In the first embodiment, HA is transported and utilized in the form of suspension. Therefore, aggregation of HA particles is suppressed, and HA can be utilized while the particle size remains small. HA with a small particle size has excellent adhesion with an object to which it is attached. HA with a small particle size can prevent the surface area of the object to be attached from decreasing.

In the first embodiment, since the concentrated HA suspension 11 can be produced by spontaneous sedimentation, the transport efficiency can be improved.

The relationship between the pH of the solution 13 in the first container 3 and the antimicrobial effects is explained below.

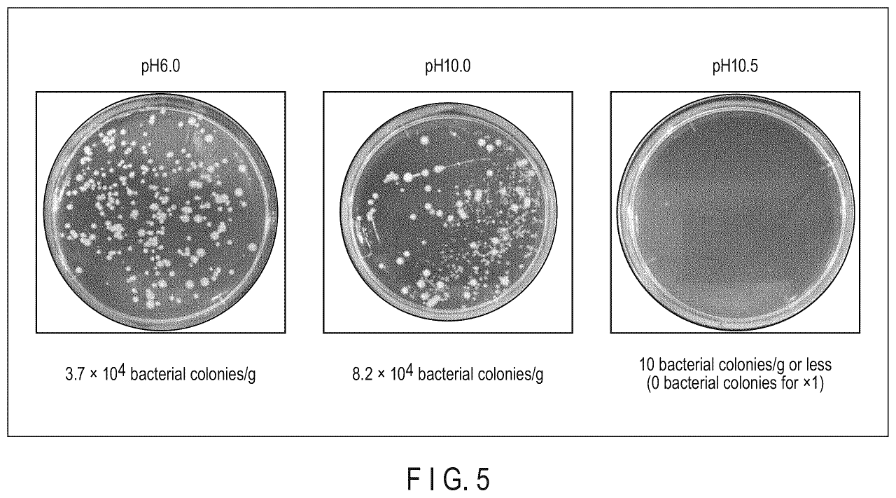

FIG. 5 shows an example of experimental results of a general viable bacteria test. In FIG. 5, the medium is a standard agar medium. The pH of the samples are 6.0, 10.0, and 10.5. 0.1 g of the solution 13 (stock solution) in the first container 3 was dropped onto the agar medium. Incubation was carried out at 35° C. for 48 hours. In FIG. 5, the number of bacterial colonies per gram is indicated for each sample.

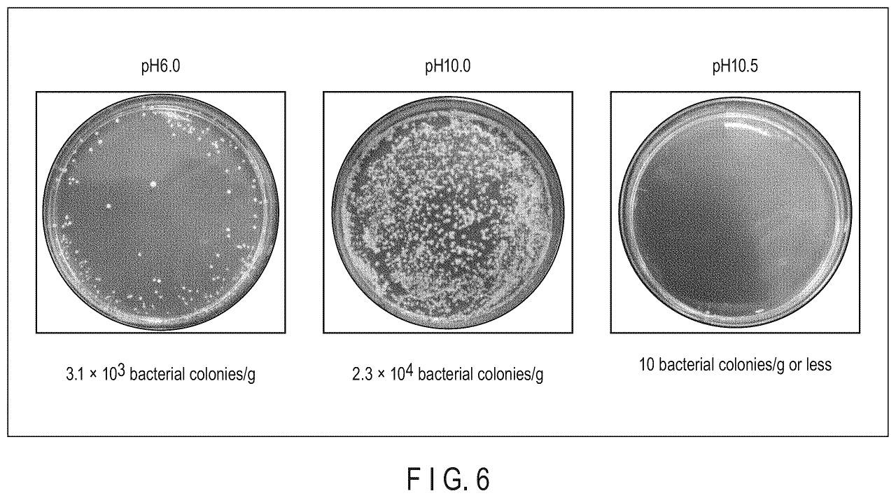

FIG. 6 shows an example of experimental results of a fungal test. In FIG. 6, the medium is a potato deoxylose agar medium to which chloramphenicol has been added. The pH of the samples are 6.0, 10.0, and 10.5. 0.1 g of the solution 13 (stock solution) in the first container 3 was dropped onto the agar medium. Incubation was carried out at 25° C. for five days. In FIG. 6, the number of bacterial colonies per gram is indicated for each sample.

In both FIG. 5 and FIG. 6, no bactericidal effect is observed at pH 10 or below, and bactericidal effect is obtained at pH 10.5 or above.

The growth limit in the alkaline region in the general viable bacteria test is pH 8 to pH 9. The growth limit in the alkaline region in the fungi test is pH 8.5. In the solution 13, when the pH is 10, sufficient bacterial count control cannot be obtained. However, in the solution 13, when the pH is 10.5 or higher, effective bacterial count control can be obtained. The solution 13 at a pH of 10.5 or higher contains a small amount of unreacted calcium hydroxide. This unreacted calcium hydroxide raises the pH of the suspension to a high value, suppressing the growth of bacteria. Thus, by controlling the pH of the solution 13, the number of bacteria can be controlled.

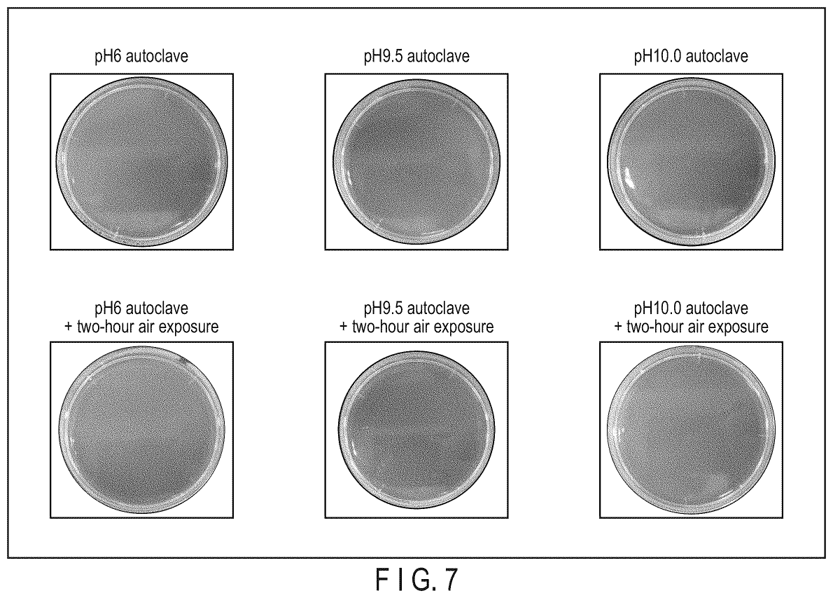

FIG. 7 shows an example of experimental results of autoclave sterilization. In FIG. 7, autoclave sterilization was carried out at 121° C. for 15 minutes. The pH of the samples are 6.0, 9.5, and 10.0. In FIG. 7, the number of bacterial colonies per gram is indicated for each sample.

By executing autoclave sterilization with respect to the solution 13, the number of bacteria can be suppressed even in a case where pH is 10 or lower. The HA in the solution 13 to which autoclave sterilization has been executed may have an increase in crystallites of about 20%. However, no aggregation of HA particles occurs. In the experiment, the solution 13 to which autoclave sterilization has been executed maintained the effect of suppressing the number of bacteria even after being exposed to air for two hours.

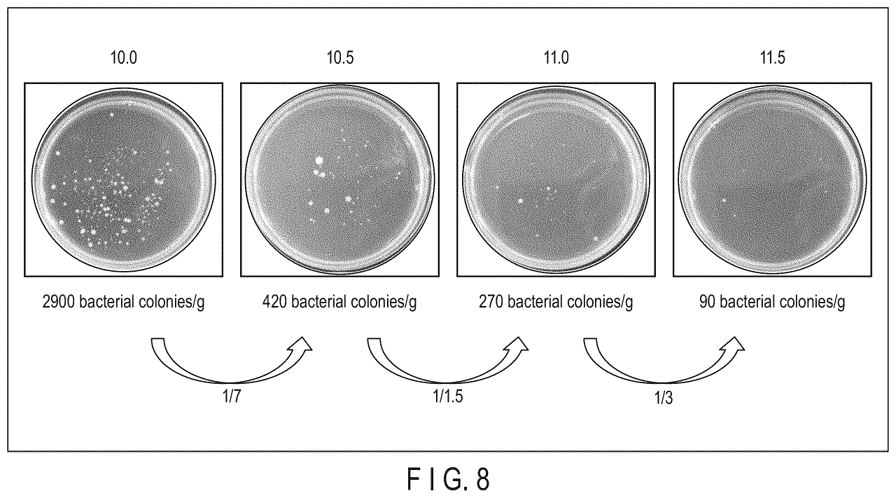

FIG. 8 shows an example of experimental results of the general viable bacteria test. In FIG. 8, the medium is a standard agar medium. The pH of the samples (synthetic pH) are 10.0, 10.5, 11.0, and 11.5. 0.1 g of the solution 13 (stock solution) in the first container 3 was dropped onto the agar medium. Incubation was performed at 35° C. for 48 hours. In FIG. 8, the number of bacterial colonies per gram is indicated for each sample.

When the pH was increased from 10 to 10.5, the number of bacterial colonies decreased by approximately 1/7. When the pH was increased from 10.5 to 11.0, the number of bacterial colonies decreased by approximately 1/1.5. When the pH was increased from 11.0 to 11.5, the number of bacterial colonies decreased by approximately 1/3.

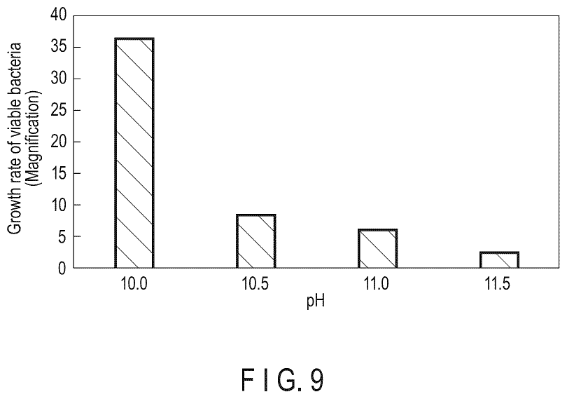

FIG. 9 is a graph showing an example of the relationship between pH and the growth rate of viable bacteria obtained based on the results of the general viable bacteria test. FIG. 9 shows the growth rate of viable bacteria for each pH from the time of concentration measurement to the time of filling the second container 11.

From FIG. 9, it can be seen that the growth rate of viable bacteria decreases significantly when the pH becomes 10.5 or higher.

From the above FIG. 8 and FIG. 9, it can be seen that the higher the pH of the solution 13 in the first container 3 is, the more effective it is in suppressing the number of viable bacteria. Specifically, when the pH of the solution 13 in the first container 3 becomes 10.5 or higher, the effect of suppressing the growth of viable bacteria increases, and the number of viable bacteria decreases significantly.

When the relationship between the pH of the solution 13 in the first container 3 and the HA yield rate was determined by experiment, the HA yield rate decreased as the pH of the solution 13 in the first container 3 increased.

Note that the HA yield rate is the ratio of the actual production amount to the calculated HA synthesis amount calculated from the raw materials.

Based on the experimental results of FIG. 8 and FIG. 9 above and the experimental results of the relationship between the pH of the solution 13 and the HA yield rate, the pH of the solution 13 that suppresses viable bacteria and increases the HA yield rate was 10 or higher and 12 or lower.

Therefore, the controller 2 according to the first embodiment executes control so that the pH of the solution 13 in the first container 3 is included in the range of 10 or higher and 12 or lower.

In the first embodiment described above, in order to maintain the particle size of HA at about nano size, the solution 13 is prepared and concentrated, the concentrated HA suspension 11 is contained in the second container 12, and the second container 12 is transported.

In the first embodiment, for example, by adjusting various conditions such as the concentration of calcium hydroxide, the concentration of phosphoric acid, the injection rate of phosphoric acid, and the temperature of the solution 13, the solution 13 containing HA with small particle size can be prepared and concentrated efficiently, and the pH of the solution 13 can be adjusted to achieve bacterial count control.

In the first embodiment, HA is shipped and utilized in a state of being contained in a suspension. Therefore, aggregation of HA particles can be suppressed, and HA with small particle size can be utilized.

In the first embodiment, concentration is performed by spontaneous sedimentation. Here, the differences between the spontaneous sedimentation used in the first embodiment and the concentration by a centrifuge, which is a comparative example, are explained. When the solution 13 is concentrated using a centrifuge, aggregation of HA particles occurs, making redispersion difficult. In contrast, in the first embodiment, since concentration is performed by spontaneous sedimentation, aggregation of HA particles can be suppressed. Concentration can also make the transportation of the HA suspension 11 more efficient and easier to handle.

Note that, in experiments, it was possible to concentrate the suspension containing nano-sized HA by about four times through spontaneous sedimentation. In a case where the particle size of HA did not have to be nano-sized, the suspension could be concentrated to about 15%.

A period required for spontaneous sedimentation varied depending on the temperature of the solution 13 in the first container 3. For example, in experiments, the number of days required to concentrate to about 3% was nine to 28 days. It was obtained from data of preparing 200 100L-class HA suspensions 11 that natural sedimentation is affected by the temperature atmosphere. From this result, in the first embodiment, the controller 2 controls the temperature of the solution 13 in the first container 3 to be at a temperature suitable for spontaneous sedimentation. In the preparation apparatus 1 according to the first embodiment, the sedimentation period could be shortened by performing spontaneous sedimentation at a temperature of 5° C. or higher and 40° C. or lower. Therefore, in the first embodiment, the solution 13 can be efficiently concentrated to prepare the HA suspension 11.

In the first embodiment, the concentrated suspension 11 is stirred by the stirrer 37 and then contained in the second container 12. Therefore, in the first embodiment, the concentration of the concentrated suspension 11 can be made uniform.

In the first embodiment, as a result of experiments, the effect of suppressing the number of bacteria was obtained by controlling the storage temperature of the solution 13 and the concentrated HA suspension 11 in the range of 2° C. or higher and 30° C. or lower. More preferably, the number of bacteria can be suppressed by controlling the storage temperature of the solution 13 and the concentrated HA suspension 11 in the range of 2° C. or higher and 20° C. or lower.

Therefore, the controller 2 and the temperature adjustment device 8 may control the temperature of the solution 13 and the concentrated HA suspension 11 in the range of 2° C. or higher and 30° C. or lower, more preferably in the range of 2° C. or higher and 20° C. or lower.

In the first embodiment, the controller 2 and the temperature adjustment device 8 may control the temperature of the solution 13 during the sedimentation period in the range of 15° C. or higher and 25° C. or lower to shorten the sedimentation period, and after the sedimentation period has elapsed, control the temperature of the solution 13 and the concentrated HA suspension 11 in the range of 2° C. or higher and 20° C. or lower for suppressing the number of bacteria. In the first embodiment, the temperature adjustment device 8 may also regulate the temperature of the concentrated HA suspension 11 in the second container 12 in addition to regulating the temperature of the solution 13 in the first container 3. In this case, the controller 2 may use the temperature adjustment device 8 to control the temperature of the concentrated HA suspension 11 in the second container 12 in the range of 2° C. or higher and 20° C. or lower.

In the first embodiment, the solution 13 is concentrated by spontaneous sedimentation. However, the concentration may be executed by centrifugation using very weak centrifugal force to maintain the dispersibility of HA in the solution 13. Furthermore, in the first embodiment, the HA suspension concentrated by spontaneous sedimentation may be further concentrated in a centrifuge using weak centrifugal force. In this case, the stirrer 37 may be used to vigorously stir the HA suspension in order to disperse the HA within the concentrated HA suspension. In a case where concentration is performed using weak centrifugal force, it is possible to suppress HA aggregation to about 6% and prepare a concentrated HA suspension.

### Second Embodiment

In a second embodiment, an apparatus and method for applying the HA suspension 11 prepared by the first embodiment above to a nonwoven fabric will be described. The second embodiment can be applied in combination with the first embodiment above.

In the second embodiment, AgHA is applied to the nonwoven fabric by immersing the nonwoven fabric in a suspension of AgHA. In the second embodiment, application is a technique utilizing wetting and solidification.

Note that, in the second embodiment, a case where AgHA is applied on the nonwoven fabric as an example of HA is described; however, the same apparatus and method can be applied to apply other HA that does not contain silver on the nonwoven fabric. Specifically, HA may contain antimicrobial heavy metals such as copper, palladium, platinum, cadmium, nickel, cobalt, zinc, manganese, thallium, lead, and mercury.

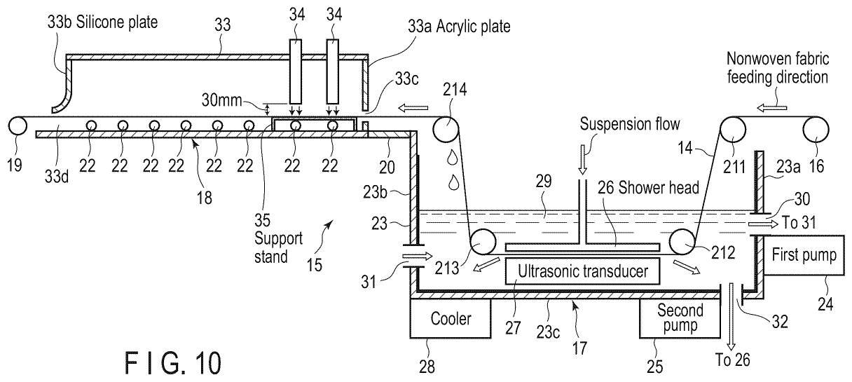

FIG. 10 is a conceptual diagram showing an example of a configuration of an application apparatus 15 for applying AgHA to a nonwoven fabric 14 according to the second embodiment. FIG. 10 exemplifies a side cross-sectional view of the application apparatus 15.

The application apparatus 15 mainly includes a feeder 16 that feeds the nonwoven fabric 1 before treatment, a tank section 17, a drying section 18, a winding section 19 that winds the nonwoven fabric 14 after treatment, and a droplet receiving section 20.

Continuous nonwoven fabric 14 is fed from the feeder 16, positioned by rollers 211 to 214 of the tank section 17 and rollers 22 of the drying section 18, and moved from right to left in FIG. 10 by the winding performed by the winding section 19. This right-to-left direction in FIG. 10 is described as a nonwoven fabric feeding direction.

The tank section 17 includes the rollers 211 to 214, a tank 23, a first pump 24, a second pump 25, a shower head 26, an ultrasonic transducer 27, and a cooler 28.

The rollers 211 and 212 guide the nonwoven fabric 14 fed from the feeder 16 into AgHA suspension liquid 29 stored in the tank 23.

The roller 212 further guides the nonwoven fabric 14 between the shower head 26 and the ultrasonic transducer 27.

The rollers 213 and 214 guide the nonwoven fabric 14 that has passed between the shower head 26 and the ultrasonic transducer 27 out of the suspension 29 stored in the tank 23.

The roller 214 further returns excess suspension contained in the nonwoven fabric 14 from the nonwoven fabric 14 to the tank 23.

The water tank 23 stores the suspension 29. The tank 23 is provided with a first outlet 30, an inlet 31, and a second outlet 32.

The first outlet 30 is provided on a first side 23a of the tank 23.

The inlet 31 is provided on a second side 23b of the tank 23. The second side 23b may be a side facing the first side 23a.

The first pump 24 discharges the suspension 29 out of the tank 23 from the first outlet 30 and allows the suspension 29 to flow into the tank 23 from the inlet 31. This causes the suspension 29 to flow within the tank 23.

The second outlet 32 is provided at the bottom 23c of the tank 23.

The second pump 25 discharges the suspension 29 out of the tank 23 from the second outlet 32 and supplies the suspension 29 to the shower head 26. This allows the suspension 29, which exists deep in the tank 23 and has a high concentration of AgHA, to be supplied to the shower head 26, causing the suspension 29 to jet out from the shower head 26 and causing the suspension 29 to flow within the tank 23.

The shower head 26 jets out the suspension 29 from a liquid jetting surface. The liquid jetting surface of the shower head 26 faces a vibrating surface of the ultrasonic transducer 27 through a gap.

The ultrasonic transducer 27 is an example of an ultrasonic wave generator. The ultrasonic transducer 27 vibrates at a high frequency and emits ultrasonic waves from an ultrasonic emission surface. The ultrasonic transducer 27 may, for example, be a device that emits powerful ultrasonic waves for cell disruption. The ultrasonic emission surface of the ultrasonic transducer 27 faces a liquid discharge surface of the shower head 26 through a gap.

In the second embodiment, the shower head 26 is placed on upper side and the ultrasonic transducer 27 is placed on lower side.

The gap between the liquid discharge surface of the shower head 26 and the vibrating surface of the ultrasonic transducer 27 is, for example, larger than the thickness of the nonwoven fabric 14 and 3 mm or less. From results of experiment, it was possible to soak the suspension 29 into the nonwoven fabric 14 even when the gap was, for example, 6.5 mm or less. By making the ultrasonic waves more powerful and/or the water flow of the suspension 29 more powerful, the gap can be applied in the range of 50 mm or less.

The nonwoven fabric 14 that has passed through the gap between the shower head 26 and the ultrasonic transducer 27 becomes soaked with the suspension 29.

The cooler 28 suppresses the temperature rise of the suspension 29 in the tank 23 due to ultrasonic waves. More specifically, the cooler 28 operates when the temperature of the suspension 29 in the tank 23 exceeds a threshold value to lower the temperature of the suspension 29.

The droplet receiving section 20 is arranged between the tank section 17 and the drying section 18. The droplet receiving section 20 receives droplets of the suspension 29 dripping from the nonwoven fabric 14.

The drying section 18 includes an enclosure 33, a plurality of rollers 22, a blowout port 34, and a support stand 35.

The surface of the enclosure 33 on the side from which the nonwoven fabric 14 is carried in may be, for example, a transparent acrylic plate 33a. By using the transparent acrylic plate 33a, an operator can easily observe the inside condition of the drying section 18. The acrylic plate 33a has an opening 33c for carrying the nonwoven fabric 14 from the outside of the enclosure 33 to the inside.

The surface of the enclosure 33 on the side from which the nonwoven fabric 14 is carried out may be, for example, a flexible silicone plate 33b. The silicone plate 33b is, for example, connected to an upper surface of the enclosure 33 only at the top, and is arranged like a hanging curtain. The operator can raise this silicone plate 33b to set and change the inside of the enclosure 33. By using the flexible silicone plate 33b in this manner, the operator can easily observe and change the inside condition of the drying section 18. The use of the flexible silicone plate 33b also allows gas such as air inside the enclosure 33 to be discharged flexibly. The silicone plate 33b has an opening 33d for carrying the nonwoven fabric 14 from the inside of the enclosure 33 to the outside.

The plurality of rollers 22 move the nonwoven fabric 14 carried in through the opening 33c formed in the acrylic plate 33a of the drying section 18 in a manner to be carried out through the opening 33d formed in the silicone plate 33b of the drying section 18.

The blowout port 34 discharges wind (e.g., warm wind) for drying the nonwoven fabric 14. In the second embodiment, the blowout port 34 discharges wind in a direction perpendicular to the plane of the nonwoven fabric 14 at the acrylic plate 33a side in the enclosure 33. More specifically, the blowout port 34 is arranged on the upper surface of the enclosure 33, on the side carrying in the nonwoven fabric 14 inside the enclosure 33, and discharges wind in a downward direction with respect to the nonwoven fabric 14. In the second embodiment, the shape of the blowout port 34 is preferably circular, for example; however, may be other shapes, such as an ellipse or a square.

On the roller 22 side of the nonwoven fabric 14 opposite to the blowout port 34 side, the support stand 35 is installed to prevent the nonwoven fabric 14 receiving the wind from being caught in the roller 22.

The support stand 35 is inside the enclosure 33 and supports the nonwoven fabric 14 on the carry-in side where the nonwoven fabric 14 receives wind.

In the second embodiment, the upper surface of the support stand 35 (the surface supporting the nonwoven fabric 14) is assumed to be net-like.

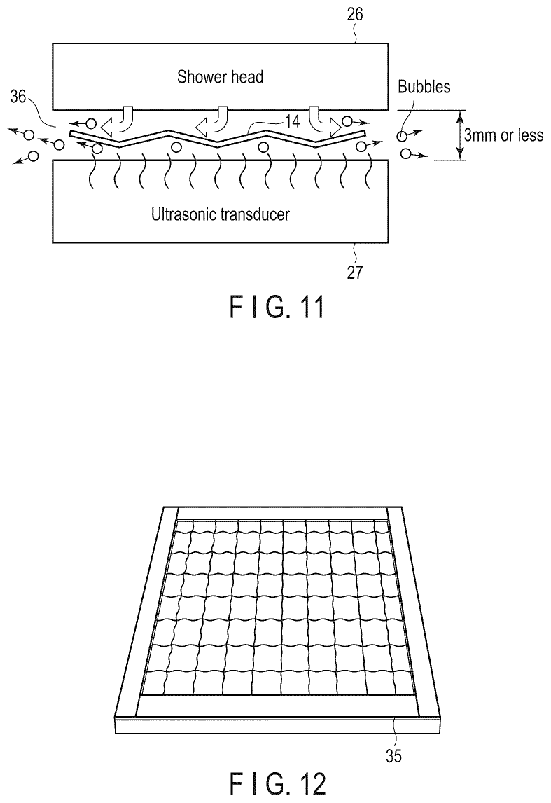

FIG. 11 is a side view showing an example of the shower head 26 and the ultrasonic transducer 27.

The shower head 26 and the ultrasonic transducer 27 are provided facing each other through a gap 36 larger than the thickness of the nonwoven fabric 14 and 3 mm or less, for example. In the second embodiment, the shower head 26 is provided on the upper side and the ultrasonic transducer 27 is provided on the lower side. However, other arrangement relationships may be applied, such as the shower head 26 on the lower side and the ultrasonic transducer 27 on the upper side.

A plurality of holes are formed in the lower surface of the shower head 26. The suspension 29 is jetted from the holes on the lower side of the shower head 26 toward the nonwoven fabric 14.

The ultrasonic transducer 27 vibrates the nonwoven fabric 14 and the suspension 29 in the gap 36 by ultrasonic waves.

The nonwoven fabric 14 immersed in the suspension 14 in the tank 23 contains air bubbles. In the second embodiment, the suspension 29 jetted from the shower head 26 is pressed against the nonwoven fabric 14. The synergistic effect of the jet of suspension 29 and the ultrasonic waves generated by the ultrasonic transducer 27 expels the air bubbles from the nonwoven fabric 14, and the hydrophobic nonwoven fabric 14 is wetted by the suspension 29.

FIG. 12 is a perspective view showing an example of the upper surface of the support stand 35.

By making the upper surface of the support stand 35 net-like, the wind discharged from the blow out port 34 can blow through the nonwoven fabric 14 more efficiently, and furthermore, the nonwoven fabric 14 can be prevented from being caught in the roller 22 under the support stand 35.

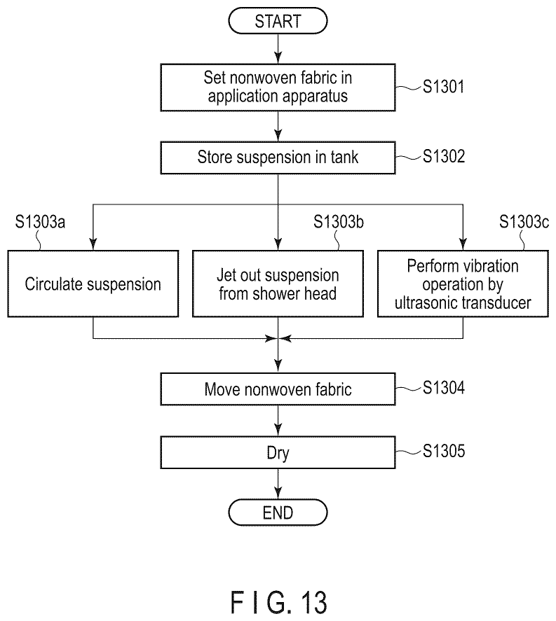

FIG. 13 is a flowchart showing an example of a method of applying AgHA to the nonwoven fabric 14 performed by the application apparatus 15 according to the second embodiment.

In step S1301, the nonwoven fabric 14 is set in the application apparatus 15 in a state where it can move in the nonwoven fabric feeding direction from the feeder 16 to the winding section 19 via the tank section 17 and the drying section 18.

In step S1302, the tank 23 stores the suspension 29.

In step S1303a, the first pump 24 circulates the suspension 29 in the tank 23.

In step S1303b, the second pump 25 supplies the suspension 29 in the tank 23 to the shower head 26, and the suspension 29 is jetted out from the shower head 26.

In step S1303c, the ultrasonic transducer 27 emits ultrasonic waves to the nonwoven fabric 14 by vibration operation to remove air bubbles from the nonwoven fabric 14.

These steps S1303a to S1303c cause the suspension 29 to soak into the hydrophobic nonwoven fabric 14.

In step S1304, the feeder 16, the rollers 211 to 214, the rollers 22, and the winding section 19 move the nonwoven fabric 14 in the nonwoven fabric feeding direction.

In step S1305, the drying section 18 dries the nonwoven fabric 14 in which the suspension 29 is soaked by the wind discharged from the blowout port 34.

The effects of the second embodiment described above will be explained.

The particle size of AgHA in the suspension 29 is smaller than the dried AgHA. In the second embodiment, AgHA having a small particle size is applied to the nonwoven fabric 14 by soaking the suspension 29 into the nonwoven fabric 14 to make it wet, and then drying it.

In a case where the nonwoven fabric 14 is hydrophobic, simply immersing the nonwoven fabric 14 in the suspension 29 may not sufficiently soak the suspension 29 into the nonwoven fabric 14, and it may not be possible to sufficiently apply AgHA to the nonwoven fabric 14.

Therefore, in the second embodiment, the nonwoven fabric 14 is vibrated by the ultrasonic vibrator 27, and the suspension 29 is jetted out from the shower head 26 toward the nonwoven fabric 14 to generate a water flow; thereby, air bubbles are expelled from within the nonwoven fabric 14 to make the nonwoven fabric 14 wet with the suspension 29, and then the nonwoven fabric 14 is quickly dried. In the second embodiment, the shower head 26 contributes to the expulsion of air bubbles in addition to the uniform jetting of the suspension 29.

This allows AgHA, which is a suspended component with a small particle size, to adhere to the nonwoven fabric 14.

In the second embodiment, the concentration of the suspension 29 in the tank 23 can be made uniform by vibrating the suspension 29 using the ultrasonic transducer 27.

In the second embodiment, the nonwoven fabric 14 passes through the gap 36 between the shower head 26 and the ultrasonic transducer 27, which face each other. By making the width of this gap greater than the thickness of the nonwoven fabric 14 and 3 mm or less, the soaking of the suspension 29 into the nonwoven fabric 14 can be accelerated.

In the second embodiment, for example, the concentration of the suspension 29 may be 0.05% or higher and 0.5% or lower. In this case, AgHA can be sufficiently adhered to the nonwoven fabric 14, and it is possible to prevent AgHA from adhering excessively to the fibers of the nonwoven fabric 14 and causing AgHA powder to fall off.

An appropriate concentration of the suspension 29 depends on the particle size.

In the case where AgHA is nano-sized, fine AgHA particles adhere more evenly to the nonwoven fabric than in the case where AgHA is micro-sized, resulting in a better application condition.

The tank 23 may be refilled with the suspension 29 so that the concentration of the suspension 29 in the tank 23 is equal to or above a predetermined value.

In the second embodiment, by adjusting the flow rate of the suspension 29 jetted from the shower head 26 and by applying ultrasonic waves to the nonwoven fabric 14 by the ultrasonic transducer 27, air bubbles in the nonwoven fabric 14 can be efficiently discharged to make the nonwoven fabric 14 wet with the suspension 29.

In the second embodiment, the case where AgHA is applied to the nonwoven fabric 14 is described, but the same apparatus and method can also be applied to a case where HA is applied to the nonwoven fabric 14 instead of AgHA.

In the second embodiment, the shower head 26 and the ultrasonic transducer 27 are used to wet the hydrophobic nonwoven fabric 14. However, in the case where the nonwoven fabric is hydrophilic, the nonwoven fabric can be made wet without using the ultrasonic transducer 27.

In the second embodiment, the nonwoven fabric 14 passes between the shower head 26 and the ultrasonic transducer 27, and excess suspension on the nonwoven fabric 14 is squeezed off by the roller 214 and returned to the water tank 23. The moderately wet nonwoven fabric 14 is then transported to the inside of the drying section 13. By spraying the suspension 29 from the shower head 26 in this manner, the efficiency of wetting the nonwoven fabric 14 can be improved and the suspension 29 in the tank 23 can be agitated.

In the second embodiment, in addition to the flow generated by the shower head 26, the suspension 29 that has flowed out from the second outlet 30 is allowed to flow in through the inlet 31, which further agitates and circulates the suspension 29 in the tank 23. This allows the concentration of the suspension 29 in the tank 23 to be made uniform, enabling the AgHA to be applied uniformly to the nonwoven fabric 14.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A hydroxyapatite suspension preparation apparatus comprising:
a container;
a first injection device that injects calcium hydroxide into the container;
a second injection device that injects phosphoric acid into a solution contained in the container;
a pH meter that measures a pH of the solution in the container;
a controller that controls concentration of the calcium hydroxide injected by the first injection device and at least one of concentration and an injection rate of the phosphoric acid injected by the second injection device so that the pH measured by the pH meter is within a predetermined range; and
a pouring device that pours hydroxyapatite suspension concentrated by spontaneous sedimentation of the solution in the container out of the container.

2. The preparation apparatus of claim 1, further comprising a stirrer that stirs the solution in the container.

3. The preparation apparatus of claim 1, wherein
the pouring device pours out a supernatant portion of the solution from the container after the spontaneous sedimentation of the solution, leaving a settled portion of the solution in the container,
the preparation apparatus further comprises a stirrer that stirs the settled portion in the container, and
the pouring device pours stirred settled portion out of the container as the concentrated hydroxyapatite suspension.

4. The preparation apparatus of claim 1, further comprising:
a thermometer that measures a temperature of the solution in the container; and
a temperature adjustment device that heats or cools the solution in the container, wherein
the controller controls the temperature adjustment device so that the temperature measured by the thermometer is within a predetermined range during the spontaneous sedimentation.

5. The preparation apparatus of claim 4, wherein the controller controls the temperature adjustment device so that the temperature is in a range of 15° C. or higher and 25° C. or lower during the spontaneous sedimentation, and controls the temperature adjustment device so that the temperature is in a range of 2° C. or higher and 20° C. or lower after the spontaneous sedimentation.

6. The preparation apparatus of claim 1, wherein the second injection device drops the phosphoric acid onto the solution containing the calcium hydroxide contained in the container.

7. The preparation apparatus of claim 1, comprising:
a tank that stores the concentrated hydroxyapatite suspension;
a shower head that jets the concentrated hydroxyapatite suspension against a nonwoven fabric in the concentrated hydroxyapatite suspension in the tank;
an ultrasonic wave generator that emits ultrasonic waves against the nonwoven fabric; and
a drying section that dries the nonwoven fabric in a wet state that is removed from the concentrated hydroxyapatite suspension in the tank.

8. A hydroxyapatite suspension preparation method comprising:
injecting calcium hydroxide into a container by a first injection device;
injecting phosphoric acid into a solution contained in the container by a second injection device;
measuring a pH of the solution in the container by a pH meter;
controlling, by a controller, concentration of the calcium hydroxide injected by the first injection device and concentration and an injection rate of the phosphoric acid injected by the second injection device so that the pH measured by the pH meter is within a predetermined range; and
pouring hydroxyapatite suspension concentrated by spontaneous sedimentation of the solution in the container out of the container by a pouring device.
